(19) United States
(12) Patent Application Publication
(10) Pub. No.: US 2004/0015212 A1
(43) Pub. Date: Jan. 22, 2004

(54) Electrotherapy stimulation device having electrode peel off detection capabilities

(75) Inventors: Hartley H. Huber, Champlin, MN (US); Lori E. Lucke, Eagan, MN (US)

Correspondence Address: Oppenheimer Wolff & Donnelly LLP, Suite 3300, 45 South Seventh Street, Minneapolis, MN, 55402-1609, US

(73) Assignee: Empi, Corp.

(21) Appl. No.: 10/160,959

(22) Filed: May 31, 2002

(51) Int. Cl. A61N 1/18

(52) U.S. Cl. 607/72

(57) ABSTRACT

Improved operating features for an electrotherapy device are provided by the use of a peel off detection system which monitors device operation and provides necessary corrective action where appropriate. More specifically, the electrotherapy device monitors the connection characteristics of the electrodes, in order to determine if acceptable connections are being maintained to the patient. In order to monitor these connections, a baseline signal measurement is made when the system is first started. Subsequent measurements are then compared to this baseline measurement, to insure that the magnitude stays within an acceptable range. If the measurement shows a non-acceptable connection condition, the electrotherapy device is shut down and appropriate warning signals are provided to the user. Where multiple output channels are used, isolation circuits are included in the feedback network in order to insure no signal coupling exists.

### BACKGROUND OF THE INVENTION

The present invention relates to an electrotherapy device having peel off detection capabilities. More specifically, the present invention relates to a controlled electrotherapy device for providing therapeutic electronic signals to a patient with the additional capability of detecting undesirable operating conditions where electrodes become disconnected or their connection characteristics significantly change. If such a condition is detected, the device can make appropriate adjustments including termination of the electrotherapy session.

Electrotherapy is an effective treatment for a number of conditions. For example, electrotherapy has generally been effective in pain management, muscle stimulation, drug delivery, and other rehabilitation treatments. All electrotherapy treatments involve the appropriate positioning of electrodes on the patient's body and the application of appropriate electrical signals. Obviously these electrodes must be connected to a signal generating device which is capable of generating and transmitting appropriate signals—most often electrical pulses.

Typically, electrodes are attached directly to the skin of the patient. A patient receiving electrotherapy treatment may be active or mobile during the therapy session, thus stressing the electrode connections. Consequently, the connected electrodes can fall off, become partially disconnected, or otherwise change their attachment characteristics. This change in connection results in a change in impedence seen by the signal generating system. The signal generating system can often times tolerate certain changes in impedance, however drastic changes are undesirable. Should drastic changes in impedance exist, it becomes very difficult to control the therapy signals being provided. Further, the change in connection characteristics creates the possibility of undesirable signals being generated, which could be unexpected and/or uncomfortable to the patient. For example, a high voltage signal could be generated (due to the high impedance created), thus creating discomfort, surprise and an aversion to further treatment sessions.

The signal generating system typically used in these electrotherapy devices is often designed to provide a constant current signal to the patient by applying constant levels of current. These particular therapies are often most effective. As can be appreciated, when constant current is being applied, the impedance of the patient will dictate the voltage level of the signal. Consequently, great variances in impedance will result in great variances in voltage. The actual voltage level however is often irrelevant, so long as it is within safe ranges. In this way, the electrotherapy system can accommodate a wide variation in impedance levels. This is beneficial as the actual human body characteristics (i.e., electrical characteristics) of many different patients can naturally vary quite vastly.

Once therapy has started, a patient applies a constant level of current. Should the electrodes become partially detached, the device continues to supply the preprogrammed constant level of current. However the partial detachment of the electrodes increases the impedance seen by the device, causing the applied voltage level to rise as the device maintains the constant level of current. This rise in voltage is undesirable as it may cause discomfort and or surprise to the patient.

Ideally, it would be beneficial to detect these undesirable operating conditions. However, due to the above-mentioned variations in impedance and related voltage often encountered, it is not practical or useful to measure the absolute voltage levels. These measurements would not allow the determination of peel off conditions based upon those absolute voltage levels. Consequently, alternative mechanisms must be developed to monitor peel off conditions.

### SUMMARY OF THE INVENTION

The present invention provides an electrotherapy device with “peel back” or “peel off” detection capabilities. Peel off generally describes a condition where the electrodes attached to the patient either physically fall off, or their connection is substantially altered (e.g., they are pulled partially off the patient). By detecting this peel off condition, the system of the present invention can subsequently control the signal generator so that no uncomfortable or unexpected signals are applied to the patient.

In operation, the signal generating device of the present invention achieves peel off detection by monitoring the output signals provided to the patient. If the output signals are as expected, the signal generating device will simply continue its therapy session. However, should unexpected signals be detected, the signal generating device determines that undesirable connection characteristics exist and will consequently shut down the device.

In order to determine if expected signal levels exist, the system will analyze relative measurements that are periodically made. The actual value or magnitude of the output signal is less relevant than its relation to a base line. In order to establish a baseline, an initiation process is utilized at the start of the therapy session. This process will develop an initial or baseline signal value for the therapy system. Subsequent signals are compared with this baseline to determine peel off conditions. As mentioned, peel off conditions cause significant changes in impedance and signal amplitude. In one embodiment of the invention, the therapy signals in the present invention are constant current signals, so variations in impedance result in changes in voltage levels. Changes in these voltage levels are indicative of the peel off conditions being detected.

The above-mentioned initiation phase is more specifically carried out by first creating an active therapy pulse when the system is turned on. Using an A-D converter within the signal generation device a voltage level for this initial signal is determined. The processor will store this baseline voltage level for future use. The processor within the signal generation device preferably includes an onboard A-D converter for carrying out this function. Further, the processor is capable of appropriately timing its sampling activities to insure that voltages are being sampled during the pulsed signal applied to the patient.

During the application of subsequent therapy signals, the controller will likewise coordinate the sampling of pulses. These samples can then be compared with the baseline value in order to determine if an undesirable change in impedance has occurred. A predefined threshold or tolerance window is established at initialization in order to more effectively carry out this function. If the change in impedance is within this window, this is determined to be an acceptable change in operating conditions and no changes are necessary. However, should the change in impedance be outside the desired tolerance window, the controller will conclude that undesirable electrode connection conditions exist, and proceed to stop the therapy session.

It is an object of the present invention to detect undesirable connection situations during electrotherapy. It is a further object of the present invention to detect changes in operating conditions and react to undesirable changes. By providing a window of acceptable conditions, the present invention is capable of anticipating expected changes and allowing continued operation.

An advantage of this system is that it also allows the signal generating system to verify that the load on the electrodes is a valid load. This prevents the device from trying to drive the output in the event that the electrodes are shorted together. It also allows the device to detect when the electrodes are not connected to the patient. In either case, the device will shutdown the output preventing possible harm to the device or the patient. Furthermore this guarantees that the device is properly connected to the patient when the device is operating and compliance data is being collected.

It is another object of the present invention to monitor the actual operating conditions and permit appropriate warnings to be generated. For example, warnings may indicate that the contacts or electrode connections should be checked, that the system may be shut down soon, or simply that the system is shut down. Further appropriate warning may be necessary or appropriate.

### BRIEF DESCRIPTION OF THE DRAWINGS

Further objects and advantages of the present invention can be seen by reading the following detailed description in conjunction with the drawings in which:

FIG. 1 is a block diagram of the therapy system which includes peel off detection capabilities;

FIG. 2 is a timing diagram illustrating a sampling timing of the present system;

FIG. 3 is a circuit diagram for a first channel of the stimulation system, which includes feedback isolation;

FIG. 4 is a circuit diagram of a second channel of the stimulation system; and

FIG. 5 is a flowchart showing the operation of the peel off detection system.

### DETAILED DESCRIPTION OF PREFERRED EMBODIMENTS

The present invention provides a device which is capable of providing electrotherapy stimulation while also including the ability to detect problems with its associated electrodes. As mentioned above, these problems can come from multiple sources including removal of the actual electrode, changes in skin conditions, etc. By providing peel off detection, preventive measures can then be taken to discontinue therapy, thus avoiding possible undesirable conditions.

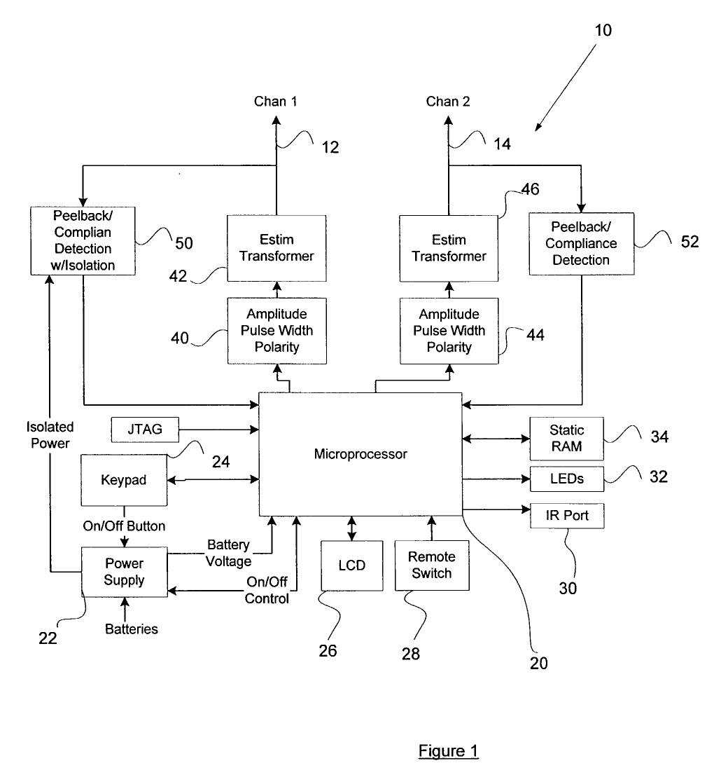

Referring now to FIG. 1, there is shown a block diagram illustrating the stimulation system 10 of the preferred embodiment. Stimulation system 10 includes a channel one output 12 and a channel two output 14, both of which are configured to be connected to the appropriate patient electrodes.

The operation of stimulation system 10 is primarily coordinated by microprocessor 20. Obviously, microprocessor 20 has a number of different components attached thereto, to support its operation. For example, a power supply 22 includes batteries to provide power to all electronic components. Other supporting components include a keypad 24, and LCD display 26, a remote switch 28, an infrared communication port 30, a number of LED indicators 32, and a static random access memory (RAM) 34. These components are relatively self-explanatory and there operation is well understood by those skilled in the art.

Microprocessor 20 coordinates all operations of the stimulation device, including the production of the actual stimulation signals. As mentioned above, stimulation device 10 includes two channels. With the exception of an isolation system, which will be further described below, the two channels are substantially identical. Consequently, the general operation of channel one will be described in detail with the understanding that channel two operates identically.

In order to control the stimulation signal, microprocessor 20 communicates with a signal generator circuit 40. The pulse width and amplitude of the actual stimulation signal will be controlled by signal generator circuit 40 based on signals received from microprocessor 20. Signal generation circuitry 40 then provides appropriate signals to a stimulation transformer 42. Based on the signals provided from control circuit 40, appropriate stimulation signals are provided at channel one output 12.

In order to provide both peel back detection and compliance monitoring capabilities, stimulation system 10 includes a detection circuit 50 which is connected between channel one output 12 and microprocessor 20.

Referring to the circuitry related to channel two, stimulation system 10 also includes a second signal generation circuit 44 and a second stimulation transformer 46, which are utilized to create the appropriate stimulation signals at channel two output 14. As is the case with channel one, channel two output 14 has connected thereto a detection circuit 52, which is also connected to microprocessor 20. First detection circuit 50 and second detection circuit 52 are also substantially similar in their operation, however, first detection circuit 50 also includes isolation circuitry, which will insure complete isolation between the two channels. As will be further described below, the use of an optocoupler provides the necessary isolation so that no coupling can exist between the two outputs.

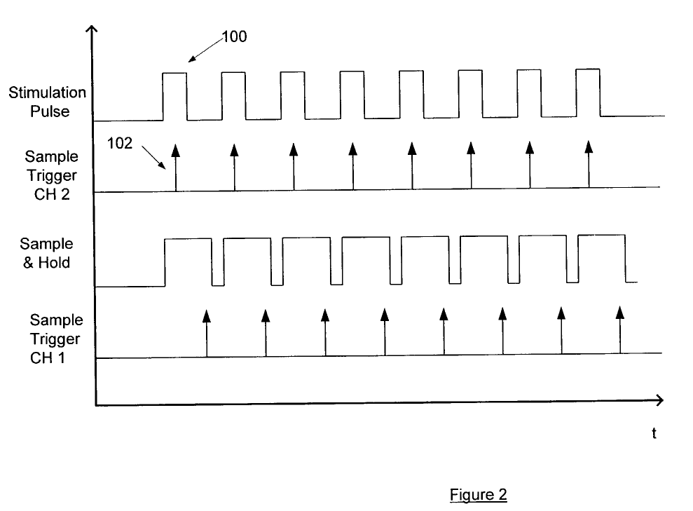

In order to appropriately control the timing and operation of the peel off detection feature, microprocessor 20 must closely coordinate stimulation signal generation with signal detection functions. First detection circuit 50 and second detection circuit 52 are both connected to microprocessor 20 at analog to digital converter inputs (not specifically shown in FIG. 1). Consequently, the signal provided to microprocessor 20 is internally converted to a digital reading. Referring to FIG. 2, the sampling operations are more easily understood. As can be seen, a sample stimulation pulse 100 is illustrated. Since microprocessor 20 controls the generation of the stimulation pulse, and can also coordinate the generation of a sampling trigger 102 for channel two. The coordination of these two signals insures that sampling is only conducted at those time periods when the pulse is expected to be driven. Based upon the results of the sampling, the peel back and compliance monitoring systems can assess the operating condition of the stimulation system 10.

As indicated above, channel one also includes isolation circuitry to insure there is no coupling between the two output channels. In order to effectively provide the required isolation, the isolation circuitry includes a dual output optocoupler and the optocoupler driver (as described in more detail below). With this circuit, it is possible to provide an isolated signal which is representative of the actual voltage at the electrodes. In practice, the isolation circuitry includes a sample and hold circuit which maintains the signal at its level for a predetermined period of time after the output is driven to allow for the microprocessor to sample the channel.

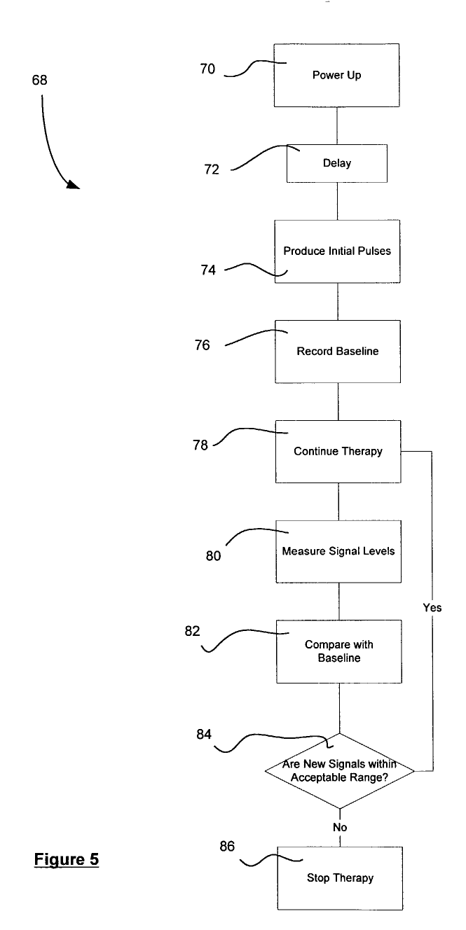

In operation, microprocessor 20 examines the relative values of the stimulation signals in order to determine if peel off problems exist. In order to accomplish this, an initialization phase is completed when the system is first powered own. This initialization phase and other parts of the system's operation, are best shown in FIG. 5 where the overall process is shown in flowchart format. First, the system is powered on in step 70. This is then followed by a delay time 72 to allow settling and ramping up of signals. During the ramp up time, the voltage is sampled and used to detect if the electrodes are shorted together or if they are attached to a patient. If shorted or unattached electrodes are detected, therapy is ended and the user is alerted to the condition. Once this ramp up delay time has elapsed, a first set of stimulation pulses are provided to the patient, based on control inputs. This is shown as step 74 in the operation process 68. Processor 20 then records the values received from first detection circuit 50 and second detection circuit 52 and stores those values as baselines in step 76. It is assumed that the electrodes and related wires are properly attached at this initialization phase, consequently these readings will create valid baselines. Throughout later therapy (following the initialization phase), the outputs from first detection circuit 50 and second detection circuit 52 are then measured and compared with these baseline values to determine if dramatic changes have taken place. In this way, the system can account for the various wide-ranging differences that might naturally exist in the use of a stimulation system. For example, the actual impedance of human skin can vary greatly, consequently an absolute value for desired voltage levels is not appropriate. This is especially true when a constant current device is utilized. However, by taking relative measurements (based on a baseline reading) drastic changes can be identified and reacted to. If system 10 is operating with an acceptable range (as determined at step 84), the system 10 will continue therapy in step 78. If the system 10 is not within acceptable levels, the therapy session will be stopped and a warning signal will be generated at step 86.

As mentioned above, the stimulation system 10 includes both a channel one output 12 and a channel two output 14. Obviously, the necessary circuitry must be connected to these outputs to create the desired therapy signals. Further, complete isolation must be provided between the two circuits in order to avoid any cross-coupling or undesired coupling affects. As discussed above in reference to FIG. 1, channel one output has connected thereto a peel back/compliance detection circuit which includes isolation. By providing isolation at this point, the necessary concerns are taken care of. Consequently, similar isolation circuitry is not necessary for channel two.

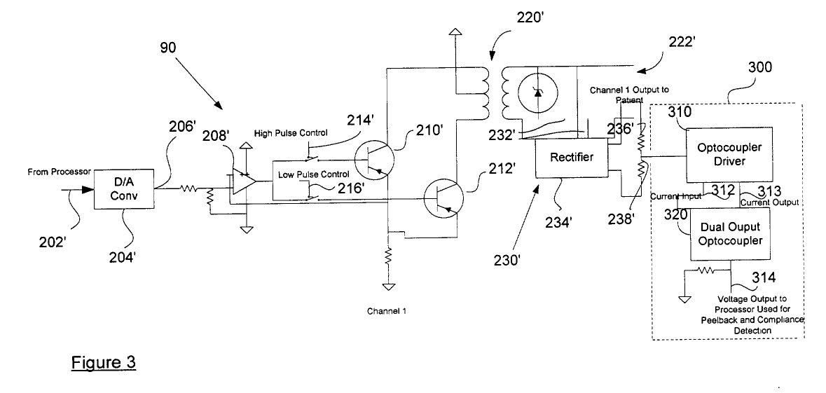

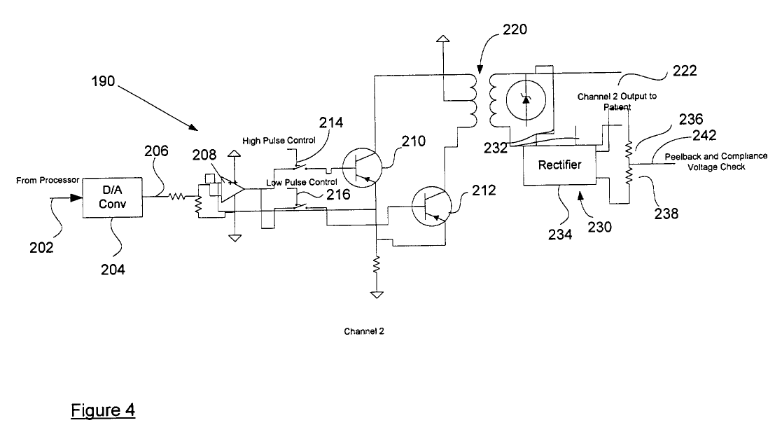

The actual details of the circuitry are shown in more detail at FIGS. 3 and 4. For simplicity, the circuitry related to channel two output 14, shown in FIG. 4, will be described first. As will be clearly understood, and will be seen by comparing FIGS. 3 and 4, much of the circuitry is identical.

Referring specifically to FIG. 4 the channel two drive circuitry 190 is shown. It can be seen that microprocessor 20 provides a first control input 202 to a D/A converter 204. Control inputs 202 is used to control the amplitude of the constant current pulses generated by channel two drive circuitry 190. D/A converter 204 provides an output signal 206 which is then provided to an amplifier 208. Amplifier 208 provides a constant current signal to both a high switching transistor 210 and a low switching transistor 212. High switching transistor 210 is controlled by a high switch control signal 214 received from microprocessor 20. Similarly, low switching transistor 212 is controlled by a low switching control signal 216, also from microprocessor 20. Both high switching transistor 210 and low switching transistor 212 are utilized to pulse a transformer 220 in the appropriate direction to produce the desired pulse polarity at the output 222 of transformer 220. The output 222 of transformer 220 is then connected to channel two output 14. In this case, channel two output 14 is a output jack capable of attachment to an electrode.

The output 222 of transformer 220 is also connected to a pulse detection circuit 230, which provides feedback to microprocessor 20 to provide peel back and compliance detection functions. More specifically, the pulse detection circuit 230 receives the output 222 from transformer 220 at a pair of input terminals 232. This signal is first rectified by rectifier 234 and then provided to a voltage divider made up of a first resistor 236 and a second resistor 238. The output is then provided at an output terminal 242 and thus transmitted to an input of microprocessor 20. The input of microprocessor 20 which receives this signal is internally connected to an onboard A/D converter capable of digitizing the analog signal level. Based on this signal level, microprocessor 20 is then capable of monitoring the voltage levels present at the output 232 of transformer 220.

Referring to FIG. 3, the channel one drive circuitry 90 is shown. Channel one drive circuitry 90 is configured to receive control signals from microprocessor 20 which will result in a constant current output. Microprocessor 20 provides all signals necessary to control the amplitude of the output pulses, the pulse width, along with the pulse rate and polarity.

It can be seen that virtually identical circuitry is utilized to generate and detect the stimulation signal in channel one. (Note that the identical circuitry shown in FIG. 3 is designated with ′ designations—e.g., programmable current generator 200′). It will be understood however, that appropriate control signals are again provided from microprocessor 20 and the channel one drive circuitry 90 has its output attached to channel one output 12. Most significantly, the output from pulse detection circuit 230′ is attached to an isolation circuit 300 to provide the necessary isolation between channel one and channel two. In summary, isolation circuit 300 includes an optocoupler driver 310, along with a dual output optocoupler 320. The output voltage at the output 222′ of transformer 220′ is first rectified using rectifier 234′. Generally speaking, the measured voltage level is then converted to a corresponding current signal which is used to drive the optocoupler. Specifically, optocoupler driver 310 will generate a current signal which is proportional to the voltage level of output 222. The dual output optocoupler provides feedback from one its outputs 313 to the optocoupler driver 310 in the form of a current signal which is used to control the input current to the optocoupler 320. The second output of the optocoupler 320 provides a current which matches the current of the first output. This current is then translated to a voltage 314 which is passed to microprocessor 20.

Referring now specifically to FIG. 3, it can be seen that the output level from detection circuit 230′ is connected to the input of a optocoupler drive 310. An output 312 from the optocoupler driver 310 is then transferred to the input of an optocoupler 320. The output from optocoupler 320 is then transferred back to microprocessor 20 for appropriate monitoring and analysis.

While the specific embodiments shown in the figures discussed above have illustrated a dual channel stimulation device, it is clearly understood that other variations are possible. For example, additional channels could easily be provided, so long as each channel has some type of microprocessor control and associated detection circuit. Further, a constant current pulse stimulation signal has been discussed above. It is clearly understood that other stimulation systems could also be provided. For example, a high volt stimulation system could be combined with a low voltage constant current stimulation to achieve a more versatile device. Further, alternative detection and stimulation circuits may also be used, where appropriate to create desired signals.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited in the particular embodiments which have been described in detail therein. Rather, reference should be made to the appended claims as indicative of the scope and content of the present invention.

What is claimed is:

1. An electrotherapy device for use in providing a stimulation signal to a patient and monitoring the device's connection characteristics so as to avoid undesirable conditions, the electrotherapy device comprising:
a stimulation signal generator having a therapy signal output attachable to the patient, the stimulation signal generator capable of producing a pulsed stimulation signal in response to a control signal, the pulsed stimulation signal having on and off periods;
a controller attached to the stimulation signal generator, the controller capable of generating the control signal so as to control the characteristics of the pulsed signal; and
a feedback connection operatively coupling the therapy signal output to the controller, wherein the controller can determine connection quality by sampling the pulsed signal during the on periods.

2. The electrotherapy device of claim 1 wherein the feedback connection is coupled to an A/D input of the controller.

3. The electrotherapy device of claim 1 wherein the connection quality is determined by comparing the sampled pulsed signal with an initialization sample, the initialization sample obtained by sampling the pulsed signal during initiation of the therapy.

4. The electrotherapy device of claim 1 wherein the stimulation signal generator includes a transformer for generating the stimulation signal in response to the control signal.

5. The electrotherapy device of claim 3 wherein the initiation of therapy includes a ramp up time prior to sampling.

6. The electrotherapy device of claim 1 further comprising:
a second stimulation signal generator attachable to the patient and connected to the controller, the second stimulation signal generator having a second therapy signal output also attachable to the patient, the second stimulation signal generator capable of producing a second pulsed stimulation signal in response to a second control signal from the controller, the second pulsed stimulation signal having on and off periods; and
a second feedback connection operatively coupling the second therapy signal output to the controller, wherein the controller can determine connection quality by sampling the pulsed signal during the on periods

7. The therapy device of claim 6 wherein the feedback connection comprises an isolation circuit attached to the therapy signal output so as to provide isolation between the therapy signal output and the second therapy signal output, the isolation circuit having an output attached to the controller.

8. The therapy device of claim 6 wherein the feedback connection comprises an isolation circuit attached to the second therapy signal output so as to provide isolation between the therapy signal output and the second therapy signal output, the isolation circuit having an output attached to the controller.

9. The electrotherapy device of claim 6 wherein the isolation circuit includes an optocoupler driver attached to the therapy signal output and an optocoupler attached to the optocoupler driver, the output of the optocoupler forming the output of the isolation circuit.

10. The electrotherapy device of claim 6 wherein the controller coordinates the sampling of the therapy signal and the second therapy signal such that sampling is only done during on periods.

11. The electrotherapy device of claim 6 wherein the connection quality for the second therapy signal generator is determined by comparing the sampled pulsed signal with an initialization sample, the initialization sample obtained by sampling the pulsed signal during initiation of the therapy.

12. The electrotherapy device of claim 6 wherein the connection quality is determined by comparing the sampled pulsed signal with an initialization sample, the initialization sample obtained by sampling the pulsed signal during initiation of the therapy.

13. A multi channel therapy device for providing a plurality of stimulation signals to a patient and for monitoring those signals to insure that undesirable conditions are avoided:
a controller for coordinating the operation of the therapy device;
a first therapy signal generator attached to the controller for producing a first therapy signal at a first therapy signal output;
a second therapy signal generator attached to the controller for producing a second therapy signal at a second therapy signal output;
an isolation circuit connecting the first therapy output to the controller while also providing electrical isolation between the first therapy output and the second therapy output;
a feedback connection attaching the second therapy signal output to the controller; and
wherein the controller will monitor the first therapy output and the second therapy output to insure that undesirable conditions are avoided.

14. The multi channel therapy device of claim 13 wherein the isolation includes an optocoupler driver and an optocoupler, wherein the magnitude of the first therapy signal is first translated to a current, and then an isolated voltage is transmitted to the controller through the optocoupler.

15. The multi channel therapy device of claim 13 further comprising an A/D converter for monitoring the therapy signals.

16. The multi channel therapy device of claim 15 wherein the controller will monitor the first and second therapy signal by generating a baseline measurement for each therapy signal and then comparing the first therapy signal and the second therapy signal with the respective baseline signal.

17. A method for providing peel off detection during the operation of an electrotherapy device, comprising:
initially attaching a plurality of electrodes to a patient, each electrode attached to the electrotherapy device;
powering up the electrotherapy device causing the creation of a plurality of initial stimulation pulses;
sampling the initial stimulation pulses to create a baseline stimulation pulse measurement and subsequently storing a baseline magnitude value representative of the magnitude of the baseline stimulation pulse measurement;
continuing to operate the electrotherapy device, thus creating a plurality of therapy pulses to provide a desired treatment to a patient;
sampling the therapy pulses to create a normal operating pulse measurement and storing a magnitude value representative of the magnitude of the therapy pulse;
comparing the magnitude value with the baseline magnitude value to determine a difference value; and
evaluating the difference value to determine if it is within a predetermined range, and if it is not within the predetermined range, shutting down the operation of the electrotherapy device.

18. The method of claim 17 wherein the electrotherapy device is a constant current device.

19. The method of claim 18 wherein the baseline stimulation pulse measurement and the normal operating pulse measurement are measurements of voltage levels.
